SEQUENCE LISTING (7 sequences)

SEQ ID NO: 1 - protein, 643 aa, Artificial
  PEPTIDE (1)..(643): synthetic construct
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFASAGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 2 - protein, 896 aa, Artificial
  PEPTIDE (1)..(896): synthetic construct
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 3 - protein, 896 aa, Artificial
  PEPTIDE (1)..(896): synthetic construct
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 4 - protein, 895 aa, Artificial
  PEPTIDE (1)..(895): synthetic construct
GADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 5 - protein, 899 aa, Artificial
  PEPTIDE (1)..(899): synthetic construct
YVEFGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 6 - protein, 642 aa, Artificial
  PEPTIDE (1)..(642): synthetic construct
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFASAGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 7 - protein, 896 aa, Artificial
  PEPTIDE (1)..(896): synthetic construct
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

(19) United States
(12) Patent Application Publication
(10) Pub. No.: US 2005/0033034 A1
(43) Pub. Date: Feb. 10, 2005

(54) ANTI-T CELL IMMUNOTOXIN FUSION PROTEIN AND ITS THERAPEUTIC USE

(76) Inventor: Gunter Engel, Weil (DE)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/496,179

(22) PCT Filed: Nov. 27, 2002
(86) PCT No.: PCT/EP02/13387

(30) Foreign Application Priority Data
Nov. 28, 2001 (GB) 0128510.5

(51) Int. Cl. A61K 39/40; C07K 16/46

(52) U.S. Cl. 530/391.1; 424/178.1

(57) ABSTRACT

A combination comprising (a) an anti-T cell immunotoxin fusion protein comprising (i) a diphtheria or Pseudomonas toxin moiety and (ii) a targeting moiety suitable for targeting the fusion protein to T cells and (b) at least one chemotherapeutic agent.

The present invention relates to a method of treating proliferative diseases, in particular leukemias or lymphomas. The invention also relates to a combination, combined preparation, pharmaceutical composition and commercial package useful in such a method.

The nature of malignant proliferative diseases like solid tumor diseases and non-solid tumor diseases is multifactorial. This presents a number of problems in providing suitable therapies which are both effective and safe. In particular, it is difficult to predict the effects of combining two or more therapeutic agents. Accordingly, there is a need for new agents and methods suitable for treating malignant proliferative diseases.

The invention provides a method of treating a malignant proliferative disease in a subject in need thereof comprising administering to the subject a combination which comprises (a) an anti-T cell immunotoxin fusion protein comprising a diptheria or Pseudomonas toxin moiety and a targeting moiety, and (b) at least one chemotherapeutic agent; a combination comprising (a) and (b) as defined above and optionally at least one pharmaceutically acceptable carrier, e.g. for simultaneous, separate or sequential use, in particular for the delay of progression or treatment of a malignant proliferative disease, especially a solid or non-solid tumor disease, such as leukemia or lymphoma; a pharmaceutical composition comprising such a combination; the use of such a combination for the preparation of a medicament for the delay of progression or treatment of a malignant proliferative disease; and a commercial package or product comprising such a combination.

More particularly, the present invention provides:

1. A combination comprising (a) an anti T cell immunotoxin fusion protein comprising (i) a diptheria or Pseudomonas toxin moiety and (ii) a targeting moiety suitable for targeting the fusion protein to T cells; and (b) at least one chemotherapeutic agent; components a) and b) may be administered simultaneously, separately or sequentially.

2. A pharmaceutical composition comprising (a) an anti-T cell immunotoxin fusion protein comprising (i) a diphtheria or Pseudomonas toxin moiety and (ii) a targeting moiety suitable for targeting the fusion protein to T cells; and (b) at least one chemotherapeutic agent.

3. A commercial package, e.g. a kit comprising (a) an anti-T cell immunotoxin fusion protein comprising (I) a diptheria or Pseudomonas toxin moiety and (ii) a targeting moiety suitable for targeting the fusion protein to T cells; and (b) at least one chematherapeutic agent; together with instructions for simultaneous, separate or sequential use thereof. In a method according to the invention.

4. Use of a combination or a pharmaceutical composition as defined above for delaying the progression of or for the treatment of a malignant proliferative disease.

5. Use of (a) an anti-T cell immunotoxin fusion protein comprising (i) a diptheria or Pseudomonas toxin moiety and (ii) a targeting moiety suitable for targeting the fusion protein to T cells; and (b) at least one chemotherapeutic agent; for the preparation of a combination or pharmaceutical composition for use in delaying the progression of or for the treatment of a malignant proliferative disease.

6. A method for delaying the progression of a malignant proliferative disease in a subject comprising administering to the subject a combination according to the invention.

A combination which comprises (a) an anti-T cell immunotoxin fusion protein comprising (i) a diptheria or Pseudomonas toxin moiety and (ii) a targeting moiety suitable for targeting the fusion protein to T cells and (b) at least one chemotherapeutic agent will be referred to hereinafter as a COMBINATION OF THE INVENTION.

According to experimental finding, in vivo the administration of a COMBINATION OF THE INVENTION, especially comprising an antineoplastic agent as combination partner (b), results in a beneficial effect, e.g. a synergistic therapeutic effect, e.g. with regard to slowing down, arresting or reversing the neoplasm formation or a longer duration of tumor response, or in other beneficial effects, e.g. less side-effects, an improved quality of life and a decreased mortality and morbidity, compared to a monotherapy applying only one of the pharmaceutically active ingredients used in the COMBINATION OF THE INVENTION, e.g. in the treatment of a tumor that is refractory to other chemotherapeutics known as anti-cancer agents. In particular, an increased up-take of the combination partner (b) in tumor tissue and tumor cells may be observed, when applied in combination with combination partner (a).

A further benefit is that lower doses of the active ingredients of the COMBINATION OF THE INVENTION can be used, for example, that the dosages need not only often be smaller but are also applied less frequently, or can be used in order to diminish the incidence of side-effects. This is in accordance with the desires and requirements of the patients to be treated.

In the combination, pharmaceutical composition or commercial package of the present invention, each of component a) and b), independently, may be present in free form or in the form of a pharmaceutically acceptable salt.

The term “combination”, as used herein defines especially a “kit of parts” in the sense that the components (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of component (a) to component (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), e.g. a more than additive effect, additional advantageous effects, less side effects, a combined therapeutical effect in a non-effective dosage of one or both of the combination partners (a) and (b).

The term “delay of progression” as used herein means administration of the combination to patients to delay tumor growth or invasiveness and/or formation, development, growth and/or spread of metastases or micrometastases. Patients may also be in a pre-stage or in an early phase of the disease. Depending on the tumor type and the particular combination used a decrease of the tumor volume can be obtained.

The term “solid tumor” especially means breast cancer, cancer of the colon and generally the GI tract, respiratory tract tumors, e.g. lung cancer, in particular small-cell lung cancer, and non-small-cell lung cancer, head and neck cancer, brain or other central nervous system tumors, oral cavity tumors, skeletal system tumors, genitourinary cancer, e.g. cervical, uterine, ovarian, testicles, prostate or bladder cancer; skin tumors, e.g. Kaposi's sarcoma. Non-solid tumors include e.g. tumors of blood or lymphatic system, e.g. leukemias and lymphomas, in particular, T cell leukemias and T cell lymphomas, Hodgkin's disease, non-Hodgkin's lymphoma, Burkitt's lymphoma, AIDS-related lymphomas, multiple myeloma, lymphoid leukemia, myeloid leukemia, acute or chronic lymphocytic leukemia, and other unspecified malignant neoplasms of lymphoid.

By “targeting moiety” it is meant any moiety which is suitable for targeting the fusion protein to T cells. Thus the targeting moiety is preferably a group, such as a polypeptide sequence, which recognises or binds to a molecule present on the surface of T cells. More preferably the targeting moiety binds specifically or selectively to T cells. In such an embodiment, the targeting moiety preferably binds to a molecule which is selectively expressed on the surface of T cells. Most preferably, the targeting moiety comprises an affinant which selectively binds to CD3, such is as an anti-CD3 antibody, particularly as disclosed in WO 01/87982 and WO 00/41474.

Component (a) as used herein includes, but is not limited to the anti-T cell immunotoxin fusion proteins comprising an immunotoxin, more specifically a diphtheria toxin moiety, and a targeting moiety, more specifically a recombinantly produced anti-CD3 antibody moiety (either monovalent divalent or multivalent). Component (a) also includes a recombinantly produced diphtheria toxin or other mutant diphtheria toxin moiety as described in WO 01/87982 or US patent application Ser. No. 09/573,797, more specifically an immunotoxin as stated above having a recombinantly produced anti-CD3 moiety from the antibody UCHT1 a antibody, more particularly an immunotoxin having the SEQ ID NOs: 1, 2, 3, 4, and 5 even more specifically DT389-sFv(UCHT1) (SEQ ID NO:6) and (Ala)dmDT390-bisFv(UCHT1*) (SEQ ID NO:7) that can be prepared and administered as described in WO 01/87982 or US patent application Ser. No. 09/573,797, the contents thereof being incorporated herein by reference.

Furthermore, component (a) as used herein includes, but is not limited to the anti-T cell immunotoxin fusion proteins comprising an immunotoxin, more specifically a pseudomonas toxin moiety, and a targeting moiety, more specifically a recombinantly produced anti-CD3 antibody moiety (either monovalent, divalent or multivalent). The combination partner (a) also includes a recombinantly produced pseudomonas toxin or other mutant pseudomonas toxin moiety as described in WO 00/41474, more specifically a pseudomonias immunotoxin as stated above having a recombinantly produced anti-CD3 moiety from the antibody UCHT1 antibody, more particularly the immunotoxin scFv(UCHT-1)-PE38 that can be prepared and administered as described in WO 00/41474, the contents thereof being incorporated herein by reference.

The pseudomonas toxin moiety is preferably derived from Pseudomonas exotoxin-A (PE) secreted by Pseudomonas aeruginosa. More preferably the pseudomonas toxin moiety comprises PE38, which is a 38 kDa fragment of PE lacking Domain 1 a (amino acid residues 1 to 250), and also lacking amino acid residues 365 to 380 numbered from the N-terminal of the mature PE polypeptide. In other words PE-38 comprises residues 251 to 364 joined to residues 381 to 613 of the mature PE polypeptide. Alternative pseudomonas toxin moieties are described on page 12 of WO 00/41474 and in U.S. Pat. No. 5,458,878, incorporated herein by reference.

In an alternative embodiment, the targeting moiety comprises interleukin-2 (IL-2) or at least a portion of the binding domain thereof, such that the fusion protein is targeted to T cells expressing the IL-2 receptor and preferably to T cells expressing the high affinity form of the IL-2 receptor. Suitable targeting moieties comprising IL-2 or fragments thereof are disclosed in WO 91/13090 and EP 0517829, the contents of which are incorporated herein by reference. In a preferred embodiment, the fusion protein comprises a diptheria toxin moiety and IL-2 or a fragment thereof, e.g. amino acid residues 2-133 of IL-2, such as DAB₄₈₆-IL-2 or DAB₃₈₉-IL-2 as described in EP 0517829.

Suitable chemotherapeutic agents as component b) include e.g. antineoplastic agents or agents used as adjuvants in cancer therapy. The term “antineoplastic agents” as used herein includes, but is not limited to aromatase inhibitors, antiestrogens, topoisomerase I inhibitors, topoisomerase 11 inhibitors, microtubule active agents, alkylating agents, antineoplastic antimetabolites, platin compounds, compounds decreasing the protein kinase activity, in particular non-receptor tyrosine kinase and further anti-anglogenic compounds, gonadorelin agonists, anti-androgens, bisphosphonates, trastuzumab and miscellaneous anti-cancer agents, e.g. 6-thioguanidine, hydroxyurea, procarbazine or bleomycin.

The term “aromatase inhibitors” as used herein relates to compounds which inhibit the estrogen production, i.e. the conversion of the substrates androstenedlone and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETEN™.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTINT™. Discodermolide can be obtained, e.g., as disclosed in U.S. Pat. No. 5,010,099.

The term “alkylating agents” as used herein includes, but is not limited to busulfan, cyclophosphamide, ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “antineoplastic antimetabolites” includes, but is not limited to 5-fluorouracil, cytarabine, fludarabine, capecitabine, gemcitabine, methotrexate and edatrexate. Capecitabine can be administered, e.g., in the form as it is marketed, e.g. under the trademark XELODA™. Gemcitabine can be administered, e.g., in the form as it is marketed, e.g. under the trademark GEMZARM™.

The term “platin compounds” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., In the form as it is marketed, e.g. under the trademark ELOXATIN™.

The term “compounds decreasing the protein kinase activity and further anti-angiogenic compounds” as used herein includes, but is not limited to compounds decreasing the activity of the epidermal growth factor (EGF) of the epidermal growth factor (EGF), the vascular endothelial growth factor (VEGF), the platelet derived growth factor (PDGF), the protein kinase C and anti-angiogenic compounds having another mechanism for their activity and/or compounds inhibiting signal transduction (e.g. non-receptor type tyrosine kinases, in particular imatinib in salt form.

Compounds which decreases the activity of VEGF are especially compounds which inhibit the VEGF receptor tyrosine kinase, compounds which inhibit a VEGF receptor and compounds binding to VEGF, and are in particular those compounds, proteins and monoclonal antibodies generically and specifically disclosed in WO 98/35958, WO 00/09495, WO 00/27820, WO 00/59509, WO 98/11223, WO 00/27819 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999; in WO 00/37502 and WO 94/10202; Angiostatimm, described by M. S. O'Reilly et al, Cell 79, 1994, 315-328; and Endostatin™, described by M. S. O'Reilly et al, Cell 88,1997,277-285;
compounds which decrease the activity of the epidermal growth factor (EGF) are especially compounds which inhibit the EGF receptor tyrosine kinase, compounds which inhibit the EGF receptor and compounds binding to EGF, and are in particular those compounds generically and specifically disclosed in WO 97/02266, EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/33980;
compounds which decreases the activity of the protein kinase C are especially those staurosporine derivatives disclosed in EP 0 296 110 (pharmaceutical preparation described in WO 00/48571) which compounds are protein kinase C inhibitors.

Further anti-anglogenic compounds are thalidomide (THALOMID), SU5416, and celecoxib (Celebrex).

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™.

Abarelix can be formulated, eg. as disclosed in U.S. Pat. No. 5,843,901.

The term “anti-androgens” as used herein includes, but is not limited to bicalutamide (CASODEX™), which can be formulated, e.g. as disclosed in U.S. Pat. No. 4,636,505.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANA™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

“Trastuzumab” can be administered, e.g., in the form as it is marketed, e.g. under the trademark HERCEPTIN™.

In each case where citations of patent applications or scientific publications are given in particular in the compound claims and the final products of the working examples, the subject-matter of the final products, the pharmaceutical preparations and the claims is hereby incorporated into the present application by reference to this publications. Comprised are likewise the corresponding racemates, enantiomers, diastereoisomers, tautomers as well as the corresponding crystal modifications where present, e.g. hydrates, solvates and polymorphs, which are disclosed therein. The compounds used as active ingredients in the combinations disclosed herein can be prepared and administered as described in the cited documents, respectively.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

It can be shown by established test models and in particular those test models described herein that a COMBINATION OF THE INVENTION results in a more effective delay of progression or treatment of a malignant proliferative disease compared to the effects observed with the single combination partners. The person skilled in the pertinent art is fully enabled to select a relevant test model to prove the hereinbefore and hereinafter mentioned therapeutic indications and beneficial effects. The pharmacological activity of a COMBINATION OF THE INVENTION may, for example, be demonstrated in a clinical study or in a test procedure as essentially described hereinafter.

Suitable clinical studies are, for example, open label non-randomized, dose escalation studies in patients with advanced solid tumors. Such studies prove in particular the synergism of the active ingredients of the COMBINATIONS OF THE INVENTION. The beneficial effects on proliferative diseases can be determined directly through the results of these studies or by changes in the study design which are known as such to a person skilled in the art. Such studies are, in particular, suitable to compare the effects of a monotherapy using the active ingredients and a COMBINATION OF THE INVENTION. Preferably, component (a) is administered with a fixed dose and the dose of component (b) is escalated until the Maximum Tolerated Dosage is reached. In a preferred embodiment of the study, each patient receives daily doses of component (a). The efficacy of the treatment can be determined in such studies, e.g., after 18 or 24 weeks by radiologic evaluation of the tumors every 6 weeks.

Alternatively, a placebo-controlled, double blind study can be used in order to prove the benefits of the COMBINATION OF THE INVENTION mentioned herein.

The COMBINATION OF THE INVENTION can also be applied in combination with surgical intervention, mild prolonged whole body hyperthermia and/or irradiation therapy.

One embodiment of the invention relates to the use of a COMBINATION OF THE INVENTION for the prevention, delay of progression or treatment of or for the preparation of a medicament for the prevention, delay of progression or treatment of malignant proliferative disease, in particular leukemias and lymphomas, even more particular T-cell leukemias and T-cell lymphomas. T-cell leukemias and lymphomas include but are not limited to T-cell prolymphotic leukemia, T-cell granular lymphotic leukemia, aggressive NK cell leukemia, hairy-cell leukemia, nasal and nasal-type NK/T cell lymphoma, mycosis fungoides and Sezary syndrome, angioimmunoblastic T-cell lymphoma, peripheral T-cell lymphoma unspecified, adult T-cell leukemia/lymphoma (HTLV1+), an a plastic large cell lymphoma, primary cutaneous OD-30 positive T-cell lymphoproliferative disorders, cutaneous T-cell lymphoma, subcutaneous panniculitis like T-cell lymphoma, intestinal T-cell lymphoma (+enteropathy), and hepatosplenic gamma/delta T-cell lymphoma.

It is one objective of this invention to provide a pharmaceutical composition comprising a quantity, which is jointly therapeutically effective against a proliferative disease comprising the COMBINATION OF THE INVENTION. In this composition, components (a) and (b) can be administered together, one after the other or separately in one combined unit dosage form or in two separate unit dosage forms. The unit dosage form may also be a fixed combination.

The pharmaceutical compositions for separate administration of components (a) and (b) and for the administration in a fixed combination, i.e. a single galenical compositions comprising at least two components (a) and (b), according to the invention can be prepared in a manner known per se and are those suitable for enteral, such as oral or rectal, or parenteral administration to mammals, including man, comprising a therapeutically effective amount of at least one pharmacologically active combination partner alone or in combination with one or more pharmaceutically acceptable carriers, especially suitable for enteral or parenteral application.

Novel pharmaceutical composition contain, for example, from about 0.1% to about 99.9%, preferably from about 20% to about 60%, of the active components. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each active compound of the COMBINATION OF THE INVENTION may be administered simultaneously or sequentially and in any order, and the components may be administered separately or as a fixed combination. For example, the method of the invention may comprise (i) administration of the component (a) in free or pharmaceutically acceptable salt form and (ii) administration of at least one component (b) in free or pharmaceutically acceptable salt form, simultaneously or sequentially in any order, in jointly therapeutically effective amounts, preferably in synergistically effective amounts, e.g. in daily dosages corresponding to the amounts described herein. The individual active components of the COMBINATION OF THE INVENTION can be administered separately at different times during the course of therapy or concurrently in divided or single combination forms. Furthermore, the term administering also encompasses the use of a pro-drug of a component that converts in vivo to the component as such. The instant invention is therefore to be understood as embracing all such regimes of simultaneous or alternating treatment and the term “administering” is to be interpreted accordingly.

The effective dosage of each component employed in the COMBINATION OF THE INVENTION may vary depending on the particular compound or pharmaceutical composition employed, the mode of administration, the condition being treated, the severity of the condition being treated. Thus, the dosage regimen of the COMBINATION OF THE INVENTION is selected in accordance with a variety of factors including the route of administration and the renal and hepatic function of the patient. A physician or clinician of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

An anti-T cell immunotoxin fusion protein comprising a diphtheria toxin moiety and a targeting moiety (e.g. (Ala)dmDT390-bisFv(UCHT1*)) is preferably administered to a human in a dosage in the range of about 1-40 μg/day, more preferably 1-30 μg/day and most preferably 1-20 p/day. Unless stated otherwise herein, the compound is preferably administered from one to four times per day over a period of 1 to 10, more preferably from 1 to 5 days. This cycle can be repeated every 1 to 4 weeks, preferably every 3 weeks.

An anti-T cell immunotoxin fusion protein comprising a pseudomonas toxin moiety and a targeting moiety (e.g. scFv(UCHT-1)-PE38) is preferably administered to a human in a dosage in the range of about 1-100 μg/day, more preferably 10-70 μg/day and most preferably 30-50 p/day. Unless stated otherwise herein, the compound is preferably administered from one to four times per day over a period of 1 to 10, more preferably from 1 to 5 days. This cycle can be repeated every 1 to 4 weeks, preferably every 3 weeks.

Non-receptor type tyrosine kinases, in particular imatinib in salt form, is preferably administered to a human in a dosage in the range of about 2.5 to 850 mg/day. Unless stated otherwise herein, the compound is preferably administered from one to four times per day.

Fadrozole may be administered orally to a human in a dosage range varying from about 0.5 to about 10 mg/day, preferably from about 1 to about 2.5 mg/day

Exemestane may be administered orally to a human in a dosage range varying from about 5 to about 200 mg/day, preferably from about 10 to about 25 mg/day, or parenterally from about 50 to 500 mg/day, preferably from about 100 to about 250 mg/day. If the drug shall be administered in a separate pharmaceutical composition, it can be administered in the form disclosed in GB 2,177,700.

Formestane may be administered parenterally to a human in a dosage range varying from about 100 to 500 mg/day, preferably from about 250 to about 300 mg/day.

Anastrozole may be administered orally to a human in a dosage range varying from about 0.25 to 20 mg/day, preferably from about 0.5 to about 2.5 mg/day.

Aminogluthemide may be administered to a human in a dosage range varying from about 200 to 500 mg/day.

Tamoxifen citrate may be administered to a human in a dosage range varying from about 10 to 40 mg/day.

Vinblastine may be administered to a human in a dosage range varying from about 1.5 to 10 mg/m² day.

Vincristine sulfate may be administered parenterally to a human in a dosage range varying from about 0.025 to 0.05 mg/kg body weight week.

Vinorelbine may be administered to a human in a dosage range varying from about 10 to 50 mg/m² day.

Etoposide phosphate may be administered to a human in a dosage range varying from about 25 to 115 mg/m² day, e.g. 56.8 or 113.6 mg/m² day.

Teniposide may be administered to a human in a dosage range varying from about 75 to 150 mg about every two weeks.

Doxorubicin may be administered to a human in a dosage range varying from about 10 to 100 mg/m² day, e.g. 25 or 50 mg/m² day.

Epirubicin may be administered to a human in a dosage range varying from about 10 to 200 mg/m² day.

Idarubicin may be administered to a human in a dosage range varying from about 0.5 to 50 mg/m² day.

Mitoxantrone may be administered to a human in a dosage range varying from about 2.5 to 25 mg/m² day.

Paclitaxel may be administered to a human in a dosage range varying from about 50 to 300 mg/m² day.

Docetaxel may be administered to a human in a dosage range varying from about 25 to 100 mg/m² day.

Cyclophosphamide may be administered to a human in a dosage range varying from about 50 to 1500 mg/m² day.

Melphalan may be administered to a human in a dosage range varying from about 0.5 to 10 mg/m² day.

5-Fluorouracil may be administered to a human in a dosage range varying from about 50 to 1000 mg/m² day, e.g. 500 mg/m² day.

Capecitabine may be administered to a human in a dosage range varying from about 10 to 1000 mg/m² day.

Gemcitabine hydrochloride may be administered to a human in a dosage range varying from about 1000 mg/week.

Methotrexate may be administered to a human in a dosage range varying from about 5 to 500 mg/m² day.

Topotecan may be administered to a human in a dosage range varying from about 1 to 5 mg/m² day.

Irinotecan may be administered to a human in a dosage range varying from about 50 to 350 mg/m² day.

Carboplatin may be administered to a human in a dosage range varying from about 200 to 400 mg/m² about every four weeks.

Cisplatin may be administered to a human in a dosage range varying from about 25 to 75 mg/m² about every three weeks.

Oxaliplatin may be administered to a human in a dosage range varying from about 50 to 85 mg/m² every two weeks.

Alendronic acid may be administered to a human in a dosage range varying from about 5 to 10 mg/day.

Clodronic acid may be administered to a human e.g. in a dosage range varying from about 750 to 1500 mg/day.

Etridonic acid may be administered to a human in a dosage range varying from about 200 to 400 mg/day.

Ibandronic acid may be administered to a human in a dosage range varying from about 1 to 4 mg every three to four weeks.

Risedronic acid may be administered to a human in a dosage range varying from about 20 to 30 mg/day.

Pamidronic acid may be administered to a human in a dosage range varying from about 15 to 90 mg every three to four weeks.

Tiludronic acid may be administered to a human in a dosage range varying from about 200 to 400 mg/day.

Trastuzumab may be administered to a human in a dosage range varying from about 1 to 4 mg/m² week.

Bicalutamide may be administered to a human in a dosage range varying from about 25 to 50 mg/m² day.

The following examples illustrate the invention described above, without intending to limit the scope of the invention in any way. The beneficial effects of the COMBINATION OF THE INVENTION can also be determined by other test models known as such to the person skilled in the pertinent art.

### EXAMPLE 1

### (Ala)dmDT390-bisFv(UCHT1*) Alone and in Combination with Imatinib, Taxol®, or Doxorubicin in Patients with Cuteanous T cell Lymphoma

Patients with cutaneous T cell lymphoma are selected. Cells are histologically assessed for II-2 receptor determined by Immunohistochemical staining using an anti-CD25 antibody and antibodies directed against the p75 component of the II-2 receptor (Foss F M et al., Curr Top Microbiol Immunol. 1998; 234: p. 63-81). Only cells histologically and immunohistochemically representative of malignant cells are assessed for 11-2R. When more than 25% of the tumor cells stained with either p55 or p75, (Ala)dmDT390-bisFv(UCHT1* treatment is begun at 9 or 18 μg/kg, i.v. over 5 min daily for 5 days and repeated every 3 weeks for a maximal period of 6 cycles. The combination partner Imatinib is administered at 100 mg/kg, p.o., every 12 h, or the combination partner Taxol® is administered at 15 mg/kg, i.v., every 48 hours, for five times, or the combination partner doxorubicin at 9 mg/kg, i.v., every 7 days.

Data points just spanning the IC50 of the agents alone or in combination are entered into the CalcuSyn program (CalcuSyn, Biosoft, Cambridge UK). This program calculates a non-exclusive combination index (Cl), whose value is indicative of the interaction of the two compounds, where Cl ˜1 represents nearly additive effects; 0.85-0.9 Indicates a slight synergism and a value below 0.85 indicates synergy.

The study indicates that combination treatment of (Ala)dmDT390-bisFv(UCHT1*) with any of the combination partner has a beneficial effect in preventing and treating cuteanous T cell lymphoma.

### EXAMPLE 2

### scFv(UCHT-1)-PE38 Alone and in Combination with Imatinib, Taxol®, or Doxorubicin in Patients with Hairy-Cell Leukemia.

Patients with hairy-cell leukemia are selected. All patients have circulating malignant cells that express CD22, adequate organ function, and an absence of high levels of neutralizing antibodies against scFv(UCHT-1)-PE38.

Between 0.2 and 4.0, mg of scFv(UCHT-1)-PE38 are diluted in 50 ml of 0.2 percent albumin in 0.9 percent sodium chloride and administered as a 30-minute intravenous Infusion every other day for a total of three doses. The combination partner Imatinib is administered at 100 mg/kg, p.o., every 12 h, or the combination patner Taxol® is administered at 15 mg/kg, i.v., every 48 hours, for five times, or the combination partner doxorubicin at 9 mg/kg, i.v., every 7 days. The disease is assessed by fluorescence-activated cell-sorter (FACS) analysis to detect the hairy-cell leukemia antigen CD22 before and after treatment.

Data points just spanning the IC50 of the agents alone or in combination are entered into the CalcuSyn program (CalcuSyn, Biosoft, Cambridge UK). This program calculates a non-exclusive combination index (CI), whose value is indicative of the interaction of the two compounds, where Cl ˜1 represents nearly additive effects; 0.85-0.9 indicates a slight synergism and a value below 0.85 indicates synergy.

The study indicates that combination treatment of scFv(UCHT-1)-PE38 with any of the combination partner has a beneficial effect in preventing and treating cuteanous T cell lymphoma.

1. A combination comprising:
(a) an anti-T cell immunotoxin fusion protein comprising (i) a diphtheria or Pseudomonas toxin moiety and (ii) a targeting moiety suitable for targeting the fusion protein to T cells; and
(b) at least one chemotherapeutic agent comprising:
(i) an antineoplastic agent selected from the group consisting of:
aromatase inhibitors, antiestrogens, topoisomerase I inhibitors, topoisomerase 11 inhibitors, microtubule active agents, busulfan, ifosfamide, melphalan, 5-fluorouracil, cytarabine, fludarabine, capecitabine, gemcitabine, edatrexate, platin compounds, compounds decreasing the protein kinase activity and further anti-angiogenic compounds, gonadorelin agonists, anti-androgens, bisphosphonates, trastuzumab, 6-thioguanidine, hydroxyurea, procarbazine and bleomycin; or
(ii) an agent used as an adjuvant in cancer therapy.

2. A pharmaceutical composition comprising:
(a) an anti-T cell immunotoxin fusion protein comprising (i) a diphtheria or Pseudomonas toxin moiety and (ii) a targeting moiety suitable for targeting the fusion protein to T cells; and
(b) at least one chemotherapeutic agent comprising:
(i) an antineoplastic agent selected from the group consisting of:
aromatase inhibitors, antiestrogens, topoisomerase I inhibitors, topoisomerase 11 inhibitors, microtubule active agents, busulfan, ifosfamide, melphalan, 5-fluorouracil, cytarabine, fludarabine, capecitabine, gemcitabine, edatrexate, platin compounds, compounds decreasing the protein kinase activity and further anti-angiogenic compounds, gonadorelin agonists, anti-androgens, bisphosphonates, trastuzumab, 6-thioguanidine, hydroxyurea, procarbazine and bleomycin; or
(ii) an agent used as an adjuvant in cancer therapy.

3. A combination according to claim 1, wherein the targeting moiety comprises an anti-CD3 antibody.

4. A combination according to claim 1, wherein the targeting moiety comprises IL-2 or at least a portion of the binding domain thereof.

5. A combination according to claim 1, wherein the anti-T cell immunotoxin fusion protein comprises a polypeptide having a sequence as defined in any one of SEQ ID NOs 1 to 7.

6-9. (canceled)

10. A method of treating a malignant proliferative disease in a subject in need thereof comprising administering to the subject a combination as defined in claim 1.

11. A method according to claim 10, wherein the malignant proliferative disease comprises a lymphoma or leukemia.

12. A method of treating a malignant proliferative disease in a subject in need thereof comprising administering to the subject a pharmaceutical composition as defined in claim 2.

13. A method according to claim 12, wherein the malignant proliferative disease comprises a lymphoma or leukemia.

14. A pharmaceutical composition according to claim 2, wherein the targeting moiety comprises an anti-CD3 antibody.

15. A pharmaceutical composition according to claim 2, wherein the targeting moiety comprises IL-2 or at least a portion of the binding domain thereof.

16. A pharmaceutical composition according to claim 2, wherein the anti-T cell immunotoxin fusion protein comprises a polypeptide having a sequence as defined in any one of SEQ ID NOs 1 to 7.

17. A combination according to claim 3, wherein the anti-T cell immunotoxin fusion protein comprises a polypeptide having a sequence as defined in any one of SEQ ID NOs 1 to 7.

18. A combination according to claim 4, wherein the anti-T cell immunotoxin fusion protein comprises a polypeptide having a sequence as defined in any one of SEQ ID NOs 1 to 7.

19. A pharmaceutical composition according to claim 14, wherein the anti-T cell immunotoxin fusion protein comprises a polypeptide having a sequence as defined in any one of SEQ ID NOs 1 to 7.

20. A pharmaceutical composition according to claim 15, wherein the anti-T cell immunotoxin fusion protein comprises a polypeptide having a sequence as defined in any one of SEQ ID NOs 1 to 7.
